(19) United States
(12) Patent Application Publication
(10) Pub. No.: US 2004/0162328 A1
(43) Pub. Date: Aug. 19, 2004

(54) Compounds of the family of 3-alkyl-(4,5-diphenyl-imidazol-1-yl) and their use as soothing agents

(76) Inventors: Jean-Baptiste Galey, Aulnay-sous-Bois (FR); Yann Mahe, Morsang-sur-Orge (FR); Maria Dalko, Gif sur Yvette (FR); Jacqueline Dumats, Villepinte (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,654

(22) PCT Filed: Apr. 24, 2002
(86) PCT No.: PCT/FR02/01413

(30) Foreign Application Priority Data
Apr. 24, 2001 (FR) 01/05498

(51) Int. Cl. C07D 233/61; A61K 31/4172

(52) U.S. Cl. 514/400; 548/335.5

(57) ABSTRACT

The invention concerns novel compounds of the family of 3-Alkyl-(4,5 diphenyl-imidazol-1-yl), their synthesis method and cosmetic, hygienic or pharmaceutical compositions containing them The invention also concerns the use, in a physiologically acceptable medium, in or for preparing a soothing composition, of at least a compound of the family of 3-Alkyl-(4,5 diphenyl-imidazol-1-yl). The invention further concerns the use, in a physiologically acceptable medium, in or for preparing a composition, a compound of the family of 3-Alkyl-(4,5 diphenyl-imidazol-1-yl), the compound and the composition being designed for soothing skin troubles such as sensitive skins, discomfort, gnawing, itching, irritations, red spots, heat and/or flush sensations, advantageously sensitive and/or irritable and/or reactive and/or allergic symptoms of the skin and/or the scalp and/or mucosa. Finally, the invention concerns a soothing cosmetic treatment using such a composition

The present invention relates to novel compounds of the 3-alkyl(4,5-diphenylimidazol-1-yl) family, to a process for synthesizing them and to cosmetic, hygiene or pharmaceutical compositions containing them.

The invention also relates to the use of at least one compound of the 3-alkyl(4,5-diphenylimidazol-1-yl) family, in a physiologically acceptable medium, in or for the preparation of a calmative composition.

The invention also relates to the use of at least one compound of the 3-alkyl(4,5-diphenylimidazol-1-yl) family, in a physiologically acceptable medium, in or for the preparation of a composition, the compound or the composition being intended to soothe skin disturbances such as sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes.

The invention also relates to a calmative cosmetic process using such a composition.

It is known that human beings are subject to phenomena of an attacking nature characterized, for example, by a sensation of skin discomfort, tautness, itching, irritation, sensations of hot skin or redness.

Novel compounds for soothing these types of attack have been sought for many years in the cosmetics industry.

Even though solutions have already been proposed, it is always advantageous to have available novel products with calmative activity, especially for minor skin disturbances, for instance sensations of inflammation, discomfort, tautness, itching, redness, a hot sensation, the symptoms of sensitive and/or irritable and/or reactive and/or intolerant skin and/or scalp and/or mucous membranes, and dry patches.

For the purposes of the present invention, the expression “skin disturbances” means sensations of inflammation, discomfort, tautness, itching, irritation, redness, a hot sensation, the symptoms of sensitive and/or irritable and/or reactive and/or intolerant skin and/or scalp and/or mucous membranes, and dry patches.

Sensitive and/or irritable and/or reactive and/or intolerant skin and/or scalp and/or mucous membranes have been defined and characterized by the Applicant in patent EP 0 680 749. They are characterized by symptoms such as, for example, subjective signs that are essentially dysaesthetic sensations, i.e. sensations experienced in an area of skin, for instance stinging, tingling, itching or pruritus, burning, inflammation, discomfort, tautness, etc.

In addition, sensitive skin is not allergic skin and the symptoms of sensitive skin are distinguished from inflammation by the absence of oedema.

The aim of the present invention is thus to provide novel products, which are readily synthetically available, which have calmative activity while at the same time not having any appreciable side effects.

It is known in the literature, especially from patent applications WO 95/03297 and WO 95/02591, that derivatives of the 5-arylimidazole type are capable of blocking the release of certain inflammatory cytokines. Similarly, 1-[3-(4-morpholinyl)propyl]-4-(4-fluorophenyl)-5-(4-pyridyl)imidazole has already been described for the same activity in the reference J. Med. Chem. 1996, 39, 3929-3937.

All these derivatives of 5-arylimidazole type have very powerful pharmacological activities that position them as medicinal products.

In addition, certain compounds of the 3-alkyl(4,5-diphenylimidazol-1-yl) family are described in patent JP 44 029 199.

The Applicant has now discovered novel compounds of the 3-alkyl(4,5-diphenylimidazol-1-yl) family corresponding to formula (I), which are synthetically readily available.

A first subject of the invention relates to novel compounds of the 3-alkyl(4,5-diphenylimidazol-1-yl) family corresponding to formula (I) below:

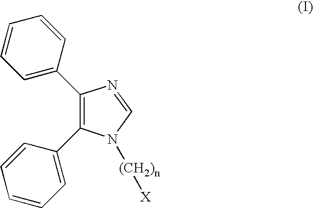

in which:

n is equal to 3, 4 or 5, advantageously 3 or 4;

X represents:

(a) a radical —NR₁R₂ in which R₁ and R₂, which may be identical or different:

represent a saturated or unsaturated, linear or branched C₁-C₈ alkyl radical optionally substituted with at least one aryl radical, which is itself optionally substituted, and/or an aralkyl radical, which is itself optionally substituted, and/or a hydroxyl radical (—OH) and/or a radical —OR₄ and/or a radical —SR₄ and/or a radical —NR₄R₅, for which R₄ and R₅ represent a linear or branched C₁-C₄ alkyl radical,

form with the nitrogen atom an optionally substituted phthalimide,

form with the nitrogen atom a 5- or 6-membered ring optionally comprising at least one other hetero atom chosen from oxygen and/or nitrogen and/or sulphur;

(b) a radical —SR₃ or a radical —SOR₃ or a radical —SO₂R₃ or a radical —COR₃ or a radical —COOR₃ in which R₃ represents:

a linear or branched C₁-C₈ alkyl radical optionally substituted with at least one aryl and/or aralkyl radical and/or a hydroxyl radical (—OH) and/or a radical —OR₄ and/or a radical —SR₄ and/or a radical —NR₄R₅, for which R₄ and R₅ represent a linear or branched C₁-C₄ alkyl radical,

an aryl radical or an aralkyl radical or a heterocycle, optionally substituted with at least one alkyl radical and/or a hydroxyl radical (—OH) and/or a radical —OR₄ and/or a radical —SR₄ and/or a radical —NR₄R₅, for which R₄ and R₅ represent a linear or branched C₁-C₄ alkyl radical.

The invention also relates to the optical and/or geometrical isomers and also the physiologically acceptable salts of the compounds corresponding to formula (I), alone or as a mixture in all proportions.

According to the invention, the expression “linear or branched C₁-C₄ and C₁-C₈ alkyl radical” means acyclic radicals derived from the removal of a hydrogen atom in the molecule of a linear or branched hydrocarbon containing from 1 to 4, or from 1 to 8, carbon atoms, and in particular methyl, ethyl, propyl, isopropyl, butyl, isobutyl, tert-butyl, pentyl, hexyl and heptyl radicals, and also the corresponding positional isomers thereof.

According to the invention, the term “heterocycle” means a sequence of atoms closed on itself and comprising at least one ring member (hetero atom) that is different from the others. According to the invention, the heterocycle may or may not be aromatic.

Preferably, the compounds of formula (I) that are preferred are those for which n is equal to 3 or 4 and X represents a radical —NR₁R₂ or a radical —SR₃ or a radical —COR₃ or a radical —COOR₃.

Advantageously, n is equal to 3 or 4 and X represents:

a radical —NR₁R₂ in which R₁ and R₂ form with the nitrogen atom a 5- or 6-membered ring, advantageously a 6-membered ring, optionally comprising at least one other hetero atom chosen from oxygen and/or nitrogen and/or sulphur, preferably an oxygen atom, and most preferably form with the nitrogen atom a morpholine,

a radical —NR₁R₂ in which R₁ and R₂ form with the nitrogen atom an optionally substituted phthalimide,

a radical —NR₁R₂ in which R₁ and R₂ represent a linear, saturated, unsubstituted C₁-C₄ alkyl radical, which are preferably identical and advantageously denote a methyl radical or an ethyl radical.

Among the compounds corresponding to formula (I), the following are most particularly preferred:

3-phthalimidepropyl(4,5-diphenylimidazol)-1-yl,

4-phthalimidebutyl(4,5-diphenylimidazol)-1-yl,

5-phthalimidepentyl(4,5-diphenylimidazol)-1-yl,

4-[3-(4,5-diphenylimidazol-1-yl)propyl]morpholine,

5-[3-(4,5-diphenylimidazol-1-yl)butyl]morpholine,

6-[3-(4,5-diphenylimidazol-1-yl)pentyl]morpholine,

4-[3-(4,5-diphenylimidazol-1-yl)propyl]thio-morpholine,

5-[3-(4,5-diphenylimidazol-1-yl)butyl]thio-morpholine,

6-[3-(4,5-diphenylimidazol-1-yl)pentyl]thio-morpholine,

4-[3-(4,5-diphenylimidazol-1-yl)propyl]piperidine,

5-[3-(4,5-diphenylimidazol-1-yl)butyl]piperidine,

6-[3-(4,5-diphenylimidazol-1-yl)pentyl]piperidine,

4-[3-(4,5-diphenylimidazol-1-yl)propyl]pyrrolidine,

5-[3-(4,5-diphenylimidazol-1-yl)butyl]pyrrolidine,

6-[3-(4,5-diphenylimidazol-1-yl)pentyl]pyrrolidine,

N,N′-diethyl-3-(4,5-diphenylimidazol-1-yl)-propylamine,

N-ethyl-N′-methyl-3-(4,5-diphenylimidazol-1-yl)-propylamine,

N,N′-dimethyl-4-(4,5-diphenylimidazol-1-yl)-butylamine,

N,N′-diethyl-4-(4,5-diphenylimidazol-1-yl)-butylamine,

N-ethyl-N′-methyl-4-(4,5-diphenylimidazol-1-yl)-butylamine,

N,N′-dimethyl-5-(4,5-diphenylimidazol-1-yl)-pentylamine,

N,N′-diethyl-5-(4,5-diphenylimidazol-1-yl)-pentylamine,

N-ethyl-N′-methyl-5-(4,5-diphenylimidazol-1-yl)-pentylamine,

N-methyl-N′-phenyl-3-(4,5-diphenylimidazol-1-yl)-propylamine,

N-ethyl-N′-phenyl-3-(4,5-diphenylimidazol-1-yl)-propylamine,

N-benzyl-N′-methyl-3-(4,5-diphenylimidazol-1-yl)-propylamine,

N-benzyl-N′-ethyl-3-(4,5-diphenylimidazol-1-yl)-propylamine,

N-methyl-N′-phenyl-4-(4,5-diphenylimidazol-1-yl)-butylamine,

N-ethyl-N′-phenyl-4-(4,5-diphenylimidazol-1-yl)-butylamine,

N-benzyl-N′-methyl-4-(4,5-diphenylimidazol-1-yl)-butylamine,

N-benzyl-N′-ethyl-4-(4,5-diphenylimidazol-1-yl)-butylamine,

N-methyl-N′-phenyl-5-(4,5-diphenylimidazol-1-yl)-pentylamine,

N-ethyl-N′-phenyl-5-(4,5-diphenylimidazol-1-yl)-pentylamine,

N-benzyl-N′-methyl-5-(4,5-diphenylimidazol-1-yl)-pentylamine,

N-benzyl-N′-ethyl-5-(4,5-diphenylimidazol-1-yl)-pentylamine,

methyl (4,5-diphenylimidazol-1-yl)propyl-3-carboxylate,

methyl (4,5-diphenylimidazol-1-yl)butyl-4-carboxylate,

methyl (4,5-diphenylimidazol-1-yl)pentyl-5-carboxylate,

ethyl (4,5-diphenylimidazol-1-yl)propyl-3-carboxylate,

ethyl (4,5-diphenylimidazol-1-yl)butyl-4-carboxylate,

ethyl (4,5-diphenylimidazol-1-yl)pentyl-5-carboxylate,

3-thiomethylpropyl(4,5-diphenylimidazol)-1-yl,

4-thiomethylbutyl(4,5-diphenylimidazol)-1-yl,

5-thiomethylpentyl(4,5-diphenylimidazol)-1-yl,

3-thioethylpropyl(4,5-diphenylimidazol)-1-yl,

4-thioethylbutyl(4,5-diphenylimidazol)-1-yl,

5-thioethylpentyl(4,5-diphenylimidazol)-1-yl,

3-thiophenylpropyl(4,5-diphenylimidazol)-1-yl,

4-thiophenylbutyl(4,5-diphenylimidazol)-1-yl,

5-thiophenylpentyl(4,5-diphenylimidazol)-1-yl,

3-thiobenzylpropyl(4,5-diphenylimidazol)-1-yl,

4-thiobenzylbutyl(4,5-diphenylimidazol)-1-yl,

5-thiobenzylpentyl(4,5-diphenylimidazol)-1-yl,

3-methyloxopropyl(4,5-diphenylimidazol)-1-yl-,

4-methyloxobutyl(4,5-diphenylimidazol)-1-yl,

5-methyloxopentyl(4,5-diphenylimidazol)-1-yl,

3-ethyloxopropyl(4,5-diphenylimidazol.)-1-yl,

4-ethyloxobutyl(4,5-diphenylimidazol)-1-yl,

5-ethyloxopentyl(4,5-diphenylimidazol)-1-yl,

3-phenyloxopropyl(4,5-diphenylimidazol)-1-yl,

4-phenyloxobutyl(4,5-diphenylimidazol)-1-yl,

5-phenyloxopentyl(4,5-diphenylimidazol)-1-yl,

3-benzyloxopropyl(4,5-diphenylimidazol)-1-yl,

4-benzyloxobutyl(4,5-diphenylimidazol)-1-yl,

5-benzyloxopentyl(4,5-diphenylimidazol)-1-yl,

3-methylsulphonepropyl(4,5-diphenylimidazol)-1-yl,

4-methylsulphonebutyl(4,5-diphenylimidazol)-1-yl,

5-methylsulphonepentyl(4, 5-diphenylimidazol) -1-yl,

3-ethylsulphonepropyl(4,5-diphenylimidazol)-1-yl,

4-ethylsulphonebutyl(4,5-diphenylimidazol)-1-yl,

5-ethylsulphonepentyl(4,5-diphenylimidazol)-1-yl,

3-phenylsulphonepropyl(4,5-diphenylimidazol)-1-yl,

4-phenylsulphonebutyl(4,5-diphenylimidazol)-1-yl,

5-phenylsulphonepentyl(4,5-diphenylimidazol)-1-yl,

3-benzylsulphonepropyl(4,5-diphenylimidazol)-1-yl,

4-benzylsulphonebutyl(4,5-diphenylimidazol)-1-yl,

5-benzylsulphonepentyl(4,5-diphenylimidazol)-1-yl,

3-methylsulphoxidepropyl(4,5-diphenylimidazol)-1-yl,

4-methylsulphoxidebutyl(4,5-diphenylimidazol)-1-yl,

5-methylsulphoxidepentyl(4,5-diphenylimidazol)-1-yl,

3-ethylsulphoxidepropyl(4,5-diphenylimidazol)-1-yl,

4-ethylsulphoxidebutyl(4,5-diphenylimidazol)-1-yl,

5-ethylsulphoxidepentyl(4,5-diphenylimidazol)-1-yl,

3-phenylsulphoxidepropyl(4,5-diphenylimidazol)-1-yl,

4-phenylsulphoxidebutyl(4,5-diphenylimidazol)-1-yl,

5-phenylsulphoxidepentyl(4,5-diphenylimidazol)-1-yl,

3-benzylsulphoxidepropyl(4,5-diphenylimidazol)-1-yl,

4-benzylsulphoxidebutyl(4,5-diphenylimidazol)-1-yl,

5-benzylsulphoxidepentyl(4,5-diphenylimidazol)-1-yl.

Advantageously, the compounds of formula (I) are chosen from the following compounds:

4-[3-(4,5-diphenylimidazol-1-yl)propyl]morpholine,

4-[3-(4,5-diphenylimidazol-1-yl)propyl]thio-morpholine,

4-[3-(4,5-diphenylimidazol-1-yl)propyl]piperidine,

methyl (4,5-diphenylimidazol-1-yl)propyl-3-carboxylate,

methyl (4,5-diphenylimidazol-1-yl)butyl-4-carboxylate,

3-phthalimidepropyl(4,5-diphenylimidazol)-1-yl,

4-phthalimidebutyl(4,5-diphenylimidazol)-1-yl.

Among these compounds, the following are most particularly preferred:

4-[3-(4,5-diphenylimidazol-1-yl)propyl]morpholine,

4-[3-(4,5-diphenylimidazol-1-yl)propyl]piperidine.

The compounds corresponding to formula (I) are obtained in a single step from 4,5-diphenyl-imidazole, which is a commercial product, and from a halo derivative.

Thus, the process for synthesizing the compounds of the 3-alkyl(4,5-diphenylimidazol-1-yl) family according to the invention has the advantage of allowing quick and easy access to the said compounds compared with the existing synthetic processes described elsewhere (WO 95/03297, WO 95/02591 and J. Med. Chem. 1996, 39, 3929), due to the fact that they are obtained in a single synthetic step.

According to the process of the invention, the formation of the compounds of the 3-alkyl(4,5-diphenylimidazol-1-yl) family of formula (I) results from the nucleophilic substitution of a halogen, derived from an alkyl halide, with the 4,5-diphenylimidazole anion.

Thus, a second subject of the invention relates to a process for preparing the compounds of formula (I) as defined above, from 4,5-diphenylimidazole.

According to a first embodiment, the process for preparing the compounds of formula (I) is characterized in that it consists essentially of the following steps:

a) dissolving 4,5-diphenylimidazole in an aprotic organic solvent preferably chosen from acetonitrile, acetone, tetrahydrofuran and dimethylformamide, advantageously acetonitrile;

b) adding an organic or mineral base, preferably a mineral base, advantageously chosen from potassium carbonate (K₂CO₃), sodium carbonate (Na₂CO₃) and calcium carbonate (Ca₂CO₃), most preferably K₂CO₃, in an amount of between 1 and 10 equivalents, preferably between 1 and 3 equivalents and advantageously 2 equivalents;

c) adding an alkyl halide in an amount of between 1 and 5 equivalents, advantageously 2 equivalents;

d) heating to a temperature of between 60° C. and 85° C., preferably to the reflux point of the solvent, for a period of between 6 hours and 48 hours, advantageously between 12 hours and 30 hours, preferably 24 hours;

e) evaporating the reaction medium to dryness under vacuum after cooling to room temperature;

f) dissolving the residue in water;

g) extracting the aqueous solution with an organic solvent chosen from ethyl acetate, dichloromethane and diethyl ether, preferably ethyl acetate;

h) drying and evaporating the organic phase to dryness;

i) optionally purifying the residue.

According to a second embodiment, the process for preparing the compounds of formula (I) is characterized in that it consists essentially of the following steps:

a) dissolving 4,5-diphenylimidazole in a dipolar aprotic solvent preferably chosen from dimethylformamide (DMF), dimethyl sulphoxide (DMSO) and dimethylacetamide (DMAc), advantageously DMF;

b) adding under an inert atmosphere an organic or mineral base, preferably a mineral base, advantageously chosen from sodium hydride (NaH) and butyllithium (BuLi), most preferably NaH, in an amount of between 0.9 and 1.5 equivalents, preferably between 1.0 and 1.1 equivalents, advantageously 1.0 equivalent;

c) stirring the reaction medium at a temperature of between 25° C. and 100° C., preferably at 50° C., for a period of between 30 minutes and 10 hours, advantageously between 1 hour and 2 hours, preferably for 1 hour;

d) adding an alkyl halide in an amount of between 1 and 3 equivalents, preferably between 1 and 2 equivalents, advantageously 1.2 equivalents;

e) adding potassium iodide or sodium iodide in an amount of between 1 and 3 equivalents, preferably between 1 and 2 equivalents, advantageously 1.2 equivalents;

f) heating at a temperature of between 25° C. and 100° C., preferably at 50° C., for a period of between 5 hours and 24 hours, advantageously between 10 hours and 20 hours, preferably for 15 hours;

g) taking up the reaction medium in an organic solvent chosen from dichloromethane and chloroform, preferably dichloromethane;

h) washing the organic phase with water and bicarbonate, drying it and then evaporating it to dryness;

j) optionally purifying the residue.

The purification methods that may optionally be carried out at the end of the processes according to the invention are performed according to standard methods used in organic synthesis.

According to the invention, the term “inert atmosphere” means argon or nitrogen, and the term “room temperature” means a temperature of between 15° C. and 25° C.

Detailed examples of the preparation of the compounds according to the invention are given later in the examples.

A third subject of the invention relates to a composition that comprises at least one of the compounds corresponding to formula (I) defined above.

Needless to say, the composition according to the invention may comprise the compounds of formula (I) alone or as mixtures in all proportions.

The amount of compounds of formula (I) contained in the composition according to the invention obviously depends on the desired effect and may thus vary within a wide range.

To give an order of magnitude, the composition of the invention may contain at least one compound of formula (I) in an amount representing from 0.001% to 20% of the total weight of the composition and preferably in an amount representing from 0.01% to 5% of the total weight of the composition.

The composition according to the invention may be intended for cosmetic or pharmaceutical and particularly dermatological application.

Preferably, the composition according to the invention is intended for cosmetic application.

The composition may be ingested or applied to the skin (to any area of body skin), the hair, the nails or mucous membranes (buccal, jugal, gingival, genital or conjunctival membranes).

Depending on the mode of administration, the composition according to the invention may be in any presentation form normally used, particularly in cosmetology.

A preferred composition according to the invention is a cosmetic composition intended for topical application.

The composition according to the invention contains a physiologically acceptable medium, i.e. a medium that is compatible with the skin, the lips, the scalp, mucous membranes, the eyes and/or the hair.

According to a fourth subject of the invention, at least one compound of the 3-alkyl(4,5-diphenylimidazol-1-yl) family corresponding to formula (I), as defined in the text hereinabove, may be combined with products with an irritant side effect commonly used in cosmetics or pharmaceutics, which products are occasionally cosmetic or pharmaceutical active agents. The presence of a compound of formula (I) in a cosmetic or pharmaceutical composition comprising a product with an irritant effect allows this irritant effect to be greatly attenuated, or even eliminated.

This also makes it possible to increase the amount of product with an irritant side effect compared with the amount of product normally used, for the purpose of improved efficacy.

Thus, the invention relates more particularly to a cosmetic composition, characterized in that it comprises, in a cosmetically or pharmaceutically acceptable medium, at least one product with an irritant side effect and at least one compound of the 3-alkyl(4,5-diphenylimidazol-1-yl) family corresponding to formula (I).

Examples of products with an irritant side effect that may be mentioned include surfactants (ionic or nonionic), preserving agents, organic solvents or active agents, for instance α-hydroxy acids (citric acid, malic acid, glycolic acid, tartaric acid, mandelic acid and lactic acid), β-hydroxy acids (salicylic acid and its derivatives), α-keto acids, β-keto acids, retinoids (retinol, retinal and retinoic acid), anthralins (dioxyanthranol), anthranoids, peroxides (especially benzoyl peroxide), minoxidil, lithium salts, antimetabolites, vitamin D and its derivatives, certain hair dyes or hair colorants (para-phenylenediamine and its derivatives, and aminophenols), fragrancing alcoholic solutions (fragrances, eaux de toilette, aftershave and deodorants), antiperspirants (certain aluminium salts), hair-removing or permanent-waving active agents (thiols) and depigmenting active agents (hydroquinone).

The Applicant has now discovered that the novel compounds of the 3-alkyl(4,5-diphenylimidazol-1-yl) family corresponding to formula (I) as defined above, 4,5-diphenyl-1H-imidazole-1-propanamine and 4,5-diphenyl-1H-imidazole-1-butanamine, have calmative activity.

Patent JP 44 029 199 reports antiirritant activity for certain compounds of the 3-alkyl(4,5-diphenylimidazol-1-yl) family.

The Applicant has been able, surprisingly and unexpectedly, to demonstrate superior antiirritant activities for the compounds according to the invention compared with those of the compounds described in patent JP 44 029 199.

Specifically, the comparative tests presented in the examples of the present invention demonstrate that the measured percentages of inhibition (inhibition of the response of superficial epidermal cells to an irritant agent by assay of interleukin-8 secreted by NHEK cells after stimulation with PMA as irritant agent) are better for the compounds according to the invention compared with the reference compounds of the prior art.

On account of this antiirritant effect, the advantage of using the compounds according to the invention as calmatives may be readily appreciated.

Thus, the present invention also relates to the use in a physiologically acceptable medium, in or for the preparation of a calmative composition, of at least one compound chosen from 4,5-diphenyl-1H-imidazole-1-propanamine, 4,5-diphenyl-1H-imidazole-1-butanamine and a compound of the 3-alkyl(4,5-di-phenylimidazol-1-yl) family corresponding to formula (I) as defined above.

In addition, the compounds of the 3-alkyl(4,5-diphenylimidazol-1-yl) family corresponding to formula (I), 4,5-diphenyl-1H-imidazole-1-propanamine and 4,5-diphenyl-1H-imidazole-1-butanamine also have the advantage of showing very little cytotoxicity (less than 20%). The evaluation of the cytotoxicity of the compounds was performed on normal human keratinocytes cultured in vitro by adding 3-(4,5-dimethylthiazol-2-yl)-2,5-diphenyl)tetrazolium bromide (MTT, 0.5 mg/ml) to the culture medium over the course of 2 hours, followed by spectrophotometric measurement of the formazan incorporated into the cells after 24 hours, according to the standard technique described by T. Mosmann (J. Immunological Methods; 65 (1983) 55-63).

Another advantage of the present invention is that of now having available compounds for soothing and/or relieving and/or gently combating skin disturbances such as sensitive skin, discomfort, tautness, itching, irritation, redness, hot sensations and/or sensations of inflammation, which makes the use of these compounds compatible in cosmetic compositions, particularly topical compositions.

The expression “physiologically acceptable medium” means a medium that is compatible with the skin and/or mucous membranes and/or the nails and/or the hair.

Another subject of the invention relates to the use, in a physiologically acceptable medium, in or for the manufacture of a composition, of at least one compound chosen from 4,5-diphenyl-1H-imidazole-1-propanamine, 4,5-diphenyl-1H-imidazole-1-butanamine and a compound of the 3-alkyl(4,5-diphenylimidazol-1-yl) family of formula (I) as defined above, the compound or the composition being intended to soothe skin disturbances chosen from sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes, particularly stinging, tingling, itching or pruritus, inflammation, discomfort and/or tautness.

Certain alopecias are associated with irritation of the scalp and/or symptoms such as discomfort, tautness, itching or pruritus, redness, hot sensations and/or sensations of inflammation. This is especially the case for androgenetic alopecia which results from a process that gives rise to irritation.

As a result, it may be appreciated that reducing the irritation of the scalp can represent a means for reducing and/or stabilizing natural hair loss in man.

Thus, another subject of the invention relates to the use, in a physiologically acceptable medium, in a cosmetic composition or for the preparation of a pharmaceutical composition, of at least one compound chosen from 4,5-diphenyl-1H-imidazole-1-propanamine, 4,5-diphenyl-1H-imidazole-1-butanamine and a compound of the 3-alkyl(4,5-diphenylimidazol-1-yl) family of formula (I) as defined above, the compound or the composition being intended to reduce and/or stabilize natural hair loss in man, advantageously androgenetic alopecia.

According to the invention, the compounds of the 3-alkyl(4,5-diphenylimidazol-1-yl) family corresponding to formula (I) that are preferably used according to the invention are those defined earlier in the text as being preferred.

Needless to say, according to the invention, the compounds of the 3-alkyl(4,5-diphenylimidazol-1-yl) family corresponding to formula (I), 4,5-diphenyl-1H-imidazole-1-propanamine and 4,5-diphenyl-1H-imidazole-1-butanamine may be used alone or as a mixture in any proportion.

In the cosmetic treatment of the skin disturbances mentioned above, in particular the symptoms of sensitive and/or irritable and/or reactive and/or intolerant skin and/or scalp and/or mucous membranes, the cosmetic composition according to the invention is to be applied to the areas to be treated of an individual suffering from at least one of the said skin disturbances and/or symptoms and is optionally left in contact for from several minutes to several hours and is optionally rinsed off; this being for uses that may be repeated or renewed over treatment periods ranging from a few days to several months or even several years.

Thus, another subject of the present invention is a cosmetic process for treating the skin and/or the scalp and/or mucous membranes, which is intended to soothe at least one of the skin disturbances and/or one of the symptoms mentioned above, characterized in that it consists in applying to the skin and/or the scalp and/or mucous membranes a cosmetic composition comprising at least one compound corresponding to formula (I), in leaving the said composition in contact with the skin and/or mucous membranes and/or the scalp, and in optionally rinsing it off.

The cosmetic treatment process of the invention advantageously applies to natural hair loss in man, and/or to sensitive and/or irritable and/or reactive and/or intolerant skin and/or scalp and/or mucous membranes.

The treatment process has the characteristics of a cosmetic process insofar as it can improve the aesthetics or comfort of the skin and/or mucous membranes and/or the scalp.

For topical application to the skin, the composition may especially be in the form of an aqueous or oily solution or a dispersion of lotion or serum type, emulsions of liquid or semi-liquid consistency of the milk type, obtained by dispersing a fatty phase in an aqueous phase (O/W) or conversely (W/O), or suspensions or emulsions of soft consistency of the aqueous or anhydrous cream or gel type, or alternatively microcapsules or microparticles or vesicular dispersions of ionic and/or nonionic type. These compositions are prepared according to the usual methods.

The composition according to the invention obviously comprises a cosmetically acceptable support and may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product, or a dispersion of oil in an aqueous phase using spherules, these spherules possibly being polymer nanoparticles such as nanospheres and nanocapsules or better still lipid vesicles of ionic and/or nonionic type.

This composition may be more or less fluid and may have the appearance of a white or coloured cream, a pomade, a milk, a lotion, a serum, a paste or a mousse. It may optionally be applied to the skin in the form of an aerosol. It may also be in solid form, for example in the form of a stick or a patch. It may be used as a care product, as a cleansing product, as a makeup product or as a simple deodorant product.

It may also be used for the hair in the form of aqueous, alcoholic or aqueous-alcoholic solutions, or in the form of creams, gels, emulsions or mousses, or alternatively in the form of aerosol compositions also comprising a pressurized propellant.

The composition according to the invention may also be a haircare composition, especially a shampoo, a hairsetting lotion, a medicated lotion, a styling cream or gel, a dye composition (especially for oxidation dyeing) optionally in the form of colouring shampoos, a restructuring lotion for the hair, a permanent-waving composition (especially a composition for the first stage of a permanent-waving operation), a lotion or gel for preventing hair loss, an antiparasitic shampoo, etc.

For the eyes, it may be in the form of drops, and for ingestion it may be in the form of capsules, granules, syrups or tablets.

The amounts of the various constituents of the compositions according to the invention are those conventionally used in the fields under consideration.

These compositions especially constitute cleansing creams, protective creams or care creams for the face, for the hands, for the feet, for the major anatomical folds or for the body (for example day creams, night creams, makeup-removing creams, foundation creams and antisun creams), fluid foundations, makeup-removing milks, protective or care body milks, aftersun milks, skincare lotions, gels or mousses, for instance cleansing lotions, antisun lotions, artificial tanning lotions, bath compositions, deodorizing compositions comprising a bactericidal agent, compositions for preventing hair loss, aftershave gels or lotions, and hair-removing creams.

The compositions according to the invention may also consist of solid preparations constituting cleansing soaps or bars.

The compositions may also be packaged in the form of an aerosol composition also comprising a pressurized propellant.

The composition may also be for buccodental use, for example a toothpaste. In this case, the composition may contain adjuvants and additives that are common for compositions for buccal use, and especially surfactants, thickeners, humectants, polishing agents such as silica, various active ingredients, for instance fluorides, in particular sodium fluoride, and optionally sweeteners, for instance sodium saccharinate.

When the composition is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in the cosmetics field. The emulsifier and co-emulsifier are present in the composition in a proportion ranging from 0.3% to 30% by weight and preferably from 0.5% to 20% by weight relative to the total weight of the composition. The emulsion may also contain lipid vesicles.

When the composition is an oily solution or gel, the fatty phase may represent more than 90% of the total weight of the composition.

In a known manner, the cosmetic composition may also contain adjuvants that are common in cosmetics, such as hydrophilic or lipophilic gelling agents, hydrophilic or lipophilic additives, preserving agents, antioxidants, solvents, fragrances, fillers, screening agents, odour absorbers and dyestuffs. The amounts of these various adjuvants are those conventionally used in cosmetics, for example from 0.01% to 10% of the total weight of the composition. Depending on their nature, these adjuvants may be introduced into the fatty phase, into the aqueous phase and/or into the lipid spherules.

As oils or waxes that may be used in the invention, mention may be made of mineral oils (liquid petroleum jelly), plant oils (liquid fraction of karite butter, sunflower oil), animal oils (perhydrosqualene), synthetic oils (purcellin oil), silicone oils or waxes (cyclomethicone), fluoro oils (perfluoropolyethers), beeswax, carnauba wax or paraffin wax. Fatty alcohols and fatty acids (stearic acid) may be added to these oils.

As examples of emulsifiers that may be used in the invention, mention may be made of glyceryl stearate, polysorbate 60 and the mixture of PEG-6/PEG-32/glycol stearate sold under the name Tefose® 63 by the company Gattefosse.

As solvents that may be used in the invention, mention may be made of lower alcohols, especially ethanol and isopropanol, and propylene glycol.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays, for instance bentones, metal salts of fatty acids, for instance aluminium stearates, hydrophobic silica, ethylcellulose and polyethylene.

The composition may contain other hydrophilic active agents, for instance proteins or protein hydrolysates, amino acids, polyols, urea, allantoin, sugars and sugar derivatives, water-soluble vitamins, plant extracts and hydroxy acids.

Lipophilic active agents that may be used include retinol (vitamin A) and its derivatives, tocopherol (vitamin E) and its derivatives, essential fatty acids, ceramides, essential oils and salicylic acid and its derivatives.

According to the invention, the composition may combine at least one compound of formula (I) with other active agents. Among these active agents that may be mentioned, for example, are:

agents for improving the activity on regrowth of the hair and/or on stopping hair loss, and which have already been described for this activity, for instance nicotinic acid esters including, especially, tocopheryl nicotinate, benzyl nicotinate and C₁-C₆ alkyl nicotinates, for instance methyl or hexyl nicotinate, pyrimidine derivatives, for instance those described by the Applicant in patent EP 0 540 629, particularly 2,4-diaminopyrimidine 3-oxide or “Aminexil”, those described in patents U.S. Pat. No. 4,139,619 and U.S. Pat. No. 4,596,812, particularly 2,4-diamino-6-piperidinopyrimidine 3-oxide or “Minoxidil”, agents for promoting regrowth of the hair, for instance those described by the Applicant in patents EP-B-0 648 488 and EP-B-0 672 406, and 5-α-reductase inhibitors, for instance those described by the Applicant in patent application EP-A-0 964 852;

agents for modifying cutaneous differentiation and/or proliferation and/or pigmentation, such as retinoic acid and its isomers, retinol and its esters, vitamin D and its derivatives, oestrogens such as oestradiol, kojic acid or hydroquinone;

antibacterial agents such as clindamycin phosphate, erythromycin or antibiotics of the tetracycline class;

antiparasitic agents, in particular metronidazole, crotamiton or pyrethroids;

antifungal agents, in particular compounds belonging to the imidazole class, such as econazole, ketoconazole or miconazole or the salts thereof, polyene compounds, such as amphotericin B, compounds of the allylamine family, such as terbinafine, or octopirox;

antiviral agents such as acyclovir;

antiinflammatory agents other than the compounds of formula (I) according to the invention, chosen, for example, from “steroidal” antiinflammatory agents such as hydrocortisone, betamethasone valerate or clobetasol propionate, or other antiinflammatory agents, for instance ibuprofen and its salts, diclofenac and its salts, acetaminophen or glycyrrhizic acid, or peptides, for instance a peptide containing the Lysine-Proline-Valine amino acid sequence described especially by the Applicant in patent EP 0 759 292; and/or prostaglandin-H synthase inhibitors (PGHS-1 and/or PGHS-2, also known as Cox-1 and Cox-2, respectively), chosen essentially from “non-steroidal” antiinflammatories (NSAIDs) such as salicylates, p-aminophenols, indols, heteroarylacetic acids, arylpropionic acids, advantageously aspirin, indomethacin, ibuprofen, Piroxicam and meloxicam as described in the documents TiPS, January 1997 (vol. 18) and DN&P 7(8), October 1994; and/or lipoxygenase inhibitors (Lox) such as hydroxamic acid and hydroxamates, alkylhydroxyamino acids, N-hydroxyurea derivatives and more particularly Zyleuton and nordihydroguaiaretic acid (NDGA) as described in the documents Biochemistry 1994, 33, 13391-13400 and Pharmaceutical Research, Vol. 9, No. 11, 1992;

anaesthetics such as lidocaine hydrochloride and its derivatives;

antipruriginous agents, for instance thenaldeine, trimeprazine or cyproheptadine;

keratolytic agents such as α- and β-hydroxycarboxylic acids or α- and β-keto carboxylic acids, and the salts, amides or esters thereof and more particularly hydroxy acids such as glycolic acid, lactic acid, salicylic acid, citric acid and fruit acids in general, and 5-n-octanoylsalicylic acid;

free-radical scavengers, such as α-tocopherol or its esters, superoxide dismutases, certain metal-chelating agents or ascorbic acid and its esters;

antiseborrhoeic agents such as progesterone;

antidandruff agents, for instance octopirox or zinc pyrithione;

antiacne agents, for instance retinoic acid or benzoyl peroxide;

extracts of plant and/or bacterial origin.

Other compounds may also be added to the above list, for instance Diazoxyde, Spiroxazone, phospholipids, for instance lecithin, linoleic acid, linolenic acid, salicylic acid, jasmonic acid and its derivatives described in French patent FR 2 581 542, for instance salicylic acid derivatives bearing an alkanoyl group containing from 2 to 12 carbon atoms in position 5 of the benzene ring, hydroxycarboxylic acids or keto carboxylic acids and the esters thereof, lactones and the corresponding salts thereof, anthralin, carotenoids, and eicosatetraenoic acid and eicosatrienoic acid or the esters and amides thereof.

According to one particular embodiment, the composition according to the invention also comprises at least one agent chosen from antibacterial agents, antiparasitic agents, antifungal agents, antiviral agents, antiinflammatory agents, antipruriginous agents, anaesthetics, keratolytic agents, free-radical scavengers, antiseborrhoeic agents, antidandruff agents, antiacne agents and/or agents for reducing cutaneous differentiation and/or proliferation and/or pigmentation, agents for improving the activity on regrowth of the hair and/or on stopping hair loss, and extracts of plant and/or bacterial origin.

It may also be envisaged for the composition comprising at least one compound as defined above to be in liposomal form, as described especially in patent application WO 94/22468 filed on 13 Oct. 1994 by the company Anti Cancer Inc. Thus, the compound encapsulated in the liposomes may be selectively delivered to the hair follicle.

Examples, which should not be considered as limiting the scope of the invention in any way, will now be given by way of illustration.

### EXAMPLE 1

### Synthesis of 4-[3-(4,5-diphenylimidazol-1-yl)-propyl]morpholine of the Following Formula

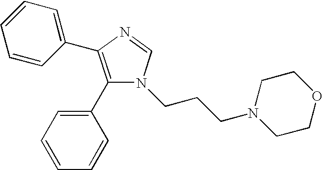

200 ml of acetonitrile, 8 g of 4,5-diphenylimidazole (36 mmol) sold by the company Acros, 10 g of K₂CO₃ (72 mmol) and then 11 g of morpholinepropyl chloride in hydrochloride form (1.5 equivalents) are placed in a three-necked flask. The mixture is refluxed for 24 hours. After cooling, the mixture is evaporated to dryness under vacuum. The mixture is dissolved in 100 ml of water and then extracted with ethyl acetate. The organic phase is washed with water and then dried over sodium sulphate. After evaporation to dryness, the oil obtained is precipitated by adding diethyl ether. The precipitate is filtered off and then recrystallized from a water/ethyl alcohol mixture (20/80).

8.6 g of product are obtained in a yield of 68%.

### Analysis

¹H NMR (400 MHz, in DMSO-d₆): δ (ppm)=1.6 (m, 2H); 2.1 (m, 6H); 3.4 (m, 4H); 3.8 (t, 2H); 7.1-7.5 (4m, 10H); 7.8 (s, 1H)

### EXAMPLES 2 TO 6

### Parallel Synthetic Scheme

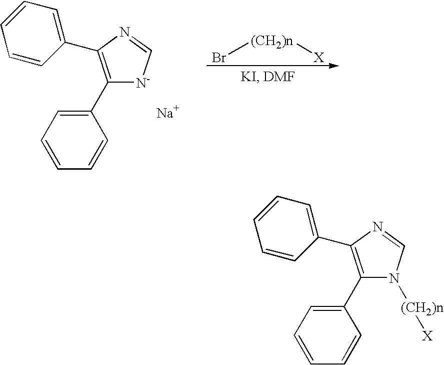

### General Protocol

A solution of the sodium salt of 4,5-diphenylimidazole is prepared by reacting 4,5-diphenylimidazole with one equivalent of sodium hydride in dimethylformamide (DMF) under argon at 50° C. for 1 hour. The solution is then divided among the tubes of a multi-well reaction block, at a rate of 3 mmol per tube. 1.2 molar equivalents of alkyl halide and of potassium iodide dissolved in DMF are then added to each tube and the mixture is heated at 50° C. for 15 hours.

Each reaction mixture is taken up in. dichloromethane and washed twice with water and bicarbonate. The organic phases are dried over sodium sulphate and evaporated to dryness.

Each residue is then analysed by HPLC coupled to mass spectrometry (electron-spray ionization).

### The Results are Collated in the Following Table

| | | FINAL PRODUCT | |
|---|---|---|---|
| | | | Result - |
| | | | analysis |
| EXAMPLES | STARTING MATERIALS | Structure-name | HPLC ESI-MS |
| 2 | 4,5-diphenylimidazole +methyl 4-butyl- carboxylate bromide | 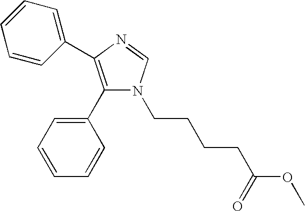 methyl (4,5-diphenyl- imidazole)-1-ylbutyl- 4-carboxylate | [M + H]⁺ = 335 detected in the majority peak |
| 3 | 3,5-diphenylimidazole +4-butylphthalimide bromide | 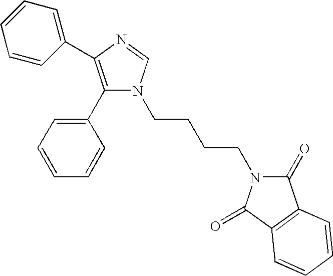 4-phthalimidebutyl- (4,5-diphenyl- imidazol)-1-yl | [M + H]⁺ = 422 detected in the majority peak |
| 4 | 4,5-diphenylimidazole +3-butylphthalimide bromide | 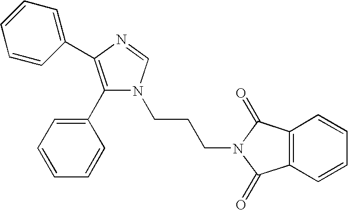 3-phthalimidepropyl- (4,5-diphenyl- imidazol)-1-yl | [M + H]⁺ = 408 detected in the majority peak |
| 5 | 4,5-diphenylimidazole +methyl 3-propyl- carboxylate bromide | 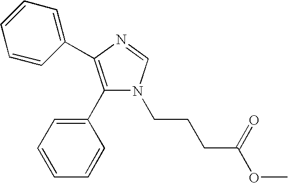 methyl (4,5-diphenyl- imidazol)-1-ylpropyl- 3-carboxylate | [M + H]⁺ = 321 detected in the majority peak |
| 6 | 4,5-diphenylimidazole +piperidinopropyl chloride | 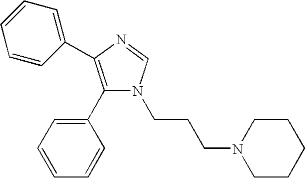 4-[3-(4,5-diphenyl- imidazole)propyl]- piperidine | [M + H]⁺ = 346 NMR and mass spectra in agreement |

### EXAMPLE 7

Inhibition of the response of superficial epidermal cells to an irritant agent by assay of the interleukin-8 secreted by normal human epidermal keratinocytes (NHEKS) after stimulation with phorbol 12-myristate 13-acetate (PMA) as irritant agent.

### Principle and Aim of the Study

This study uses the test developed by Wilmer [Wilmer, J. L. et al., J. invest. Dermatol., 102: 915-922 (1994)]. This test allows the evaluation of the antiirritant potential of various molecules, on a human keratinocyte (NHEK) cell line. In this test, an irritant situation is mimicked by exacerbating the production of IL-8 of the NHEKs by adding PMA to the culture medium, IL-8 being involved in initiating the keratinocyte irritation. The antiirritant effect of a molecule is then measured by means of its inhibitory action with respect to this exacerbated production.

### Experimental Conditions

Normal human epidermal keratinocytes (NHEKs) sold by the company Clonetics and distributed in France by the company TEBU are incubated in the presence of 160 nM of PMA for 24 hours at 37° C. In response to the PMA, the production of the chemotactic agent interleukin-8 (IL-8) is measured by enzymatic assay using an assay kit (Elisa D8050) sold by the company R&D. The absorbance values are measured using a microplate reader (MRX/Dynatech) according to the procedures supplied with the assay kit. To evaluate the antiirritant protective effect of the various compounds, the production of the chemotactic marker IL-8 by the human keratinocytes is measured in the presence of various concentrations of the test compound.

The results are expressed as a percentage of the control values after subtracting the background noise and as a percentage of inhibition of these values obtained in the presence of the compounds.

### Results

STUDY No. 1

| | | % inhibition of | |
|---|---|---|---|
| Test compounds | | secretion of IL-8 | |
| (at 1 and 10 μM) | Structure | (at 1 μM) | (at 10 μM) |
| Compound of Example 1 4-[3-(4,5-diphenylimidazol- 1-yl)propyl]- morpholine | 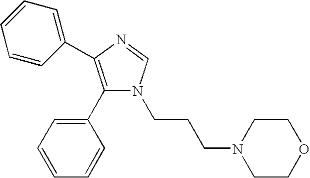 | 86 | 95 |

STUDY No. 2 (Comparative Tests)

| | | % inhibition |
|---|---|---|
| Test compounds | | of secretion |
| (at 10 μM) | Structure | of IL-8 |
| Compound of Example 1 4-[3-(4,5-diphenyl- imidazol-1-yl)- propyl]morpholine | 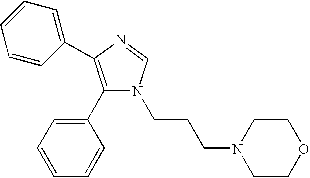 | 35 |
| Compound of Example 2 methyl (4,5-diphenyl- imidazol)-1-ylbutyl-4- carboxylate | 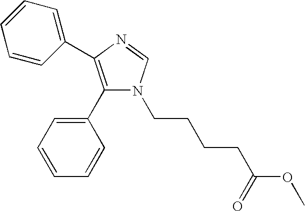 | 10 |
| Compound of Example 3 4-phthalimidebutyl- (4,5-diphenylimidazol)- 1-yl | 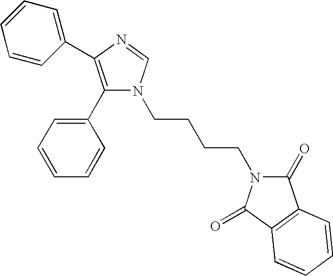 | 39 |
| Compound of Example 4 3-phthalimidepropyl- (4,5-diphenylimidazol)-1-yl | 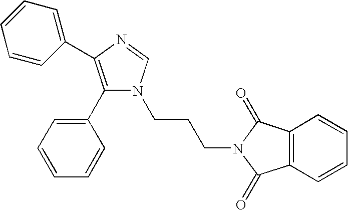 | 37 |
| Compound of Example 5 methyl (4,5-diphenyl- imidazol)-1-ylpropyl]- 3-carboxylate | 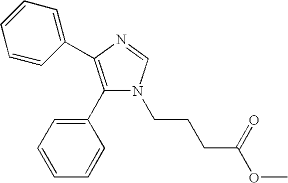 | 22 |
| Compound of Example 6 4-[3-(4,5-diphenyl- imidazole)propyl]- piperidine | 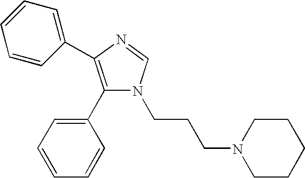 | 41 |
| JP 44 029 199 3-[3-(4,5-diphenyl- imidazole)ethyl]- piperidine | 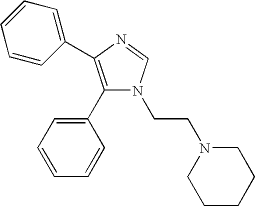 | <10 |

The results of the comparative tests show that the measured percentages of inhibition (inhibition of the response of the superficial epidermal cells to an irritant agent by assay of the interleukin-8 secreted by NHEK cells after stimulation with PMA as irritant agent) are better for the compounds according to the invention compared with the reference compounds of the prior art (JP 44 029 199).

These results thus demonstrate an anti-irritant effect of the compounds according to the invention that is better than that of the reference compounds of the prior art. As a result of this anti-irritant effect, the advantage of using the compounds according to the invention as calmatives may be readily appreciated.

The difference in the results obtained for the compound of Example 1 between study No. 1 and study No. 2 depends on the origin of the NHEK cells, the sensitivity of which may vary from one batch to another.

All the compounds of study No. 2 (Comparative Tests) were tested on the same batch of NHEK cells and under the same experimental conditions. Thus, the percentages of inhibition of the compounds of study No. 2 may be compared with each other.

### EXAMPLE 8

### Compositions

These compositions were obtained by simple mixing of the various components.

Lotion:

| 4-[3-(4,5-Diphenylimidazol-1-yl)propyl]- | | 0.5% |
|---|---|---|
| morpholine | | |
| Propylene glycol | | 10.0% |
| Isopropyl alcohol | qs | 100% |

1 ml of this lotion is applied to the scalp, at a frequency of once or twice a day.

Gel:

| Chimexane NS ® | | 1.8% |
|---|---|---|
| Monosodium stearoylglutamate | | 0.2% |
| 4-[3-(4,5-Diphenylimidazol-1-yl)propyl]- | | 1.0% |
| piperidine | | |
| Carbomer | | 0.2% |
| Triethanolamine | qs | pH = 7 |
| Preserving agents | qs | |
| Fragrances | qs | |
| Demineralized water | qs | 100% |

This gel is applied to the skin, once or twice a day.

Anti-irritant lotion for preventing hair loss:

| 4-[3-(4,5-Diphenylimidazol-1-yl)propyl]- | | 1.0% |
|---|---|---|
| morpholine | | |
| Propylene glycol | | 30.0% |
| Ethyl alcohol | | 40.5% |
| Water | qs | 100% |

This lotion is applied once or twice a day, at a rate of 1 ml per application.

Thickened lotion:

| 4-Phthalimidebutyl(4,5-diphenyl-imidazol)-1-yl | | 1.0% |
|---|---|---|
| Kawaine | | 2.0% |
| Hydroxypropylcellulose sold by the company | | 3.5% |
| Hercules under the name Klucel G ® | | |
| Ethyl alcohol | qs | 100% |

This thickened lotion is applied once or twice a day.

Lotion:

| Chimexane NL ® | | 0.50% |
|---|---|---|
| Cholesterol | | 0.40% |
| Monosodium stearoylglutamate | | 0.05% |
| 3-Phthalimidepropyl(4,5-diphenylimidazol)-1-yl | | 0.50% |
| Preserving agents | qs | |
| Colorants | qs | |
| Fragrance | qs | |
| Demineralized water | qs | 100% |

This lotion is applied once or twice a day, at a rate of 1 ml per application.

Lotion:

| 4-[3-(4,5-Diphenylimidazol-1-yl)propyl]- | | 0.1% |
|---|---|---|
| morpholine | | |
| Propylene glycol monomethyl ether sold under the name | | 20.0% |
| Dowanol PM ® by the company Dow Chemical | | |
| Hydroxypropylcellulose sold by the company Hercules | | 3.0% |
| under the name Klucel G ® | | |
| Ethyl alcohol | | 40.0% |
| Water | qs | 100% |

This thickened lotion is applied at a rate of 1 ml per application.

Day cream:

| 4-[3-(4,5-Diphenylimidazol-1-yl) | | 1.0% |
|---|---|---|
| propyl]-piperidine | | |
| Sucrose stearate | | 4.0% |
| Stearyl alcohol | | 2.0% |
| Cyclohexasiloxane | | 9.0% |
| Mineral oil | | 4.0% |
| Glycerol | | 5.0% |
| Xanthan gum | | 0.3% |
| Carbomer | | 0.5% |
| Preserving agents | | 0.3% |
| Fragrance | | 0.3% |
| Water | qs | 100% |

Care fluid:

| 4-[3-(4,5-Diphenylimidazol-1-yl)- | | 1.0% |
|---|---|---|
| propyl] morpholine | | |
| Stearyl alcohol | | 0.4% |
| Sorbitan stearate | | 1.5% |
| Glycerol | | 5.0% |
| Xanthan gum | | 0.2% |
| Carborner | | 0.1% |
| Cyclohexasiloxane | | 7.0% |
| Preserving agents | | 0.3% |
| Fragrance | | 0.2% |
| Water | qs | 100% |

Lotion:

| Methyl (4,5-diphenylimidazol)-1-yl-propyl-3- | | 0.5% |
|---|---|---|
| carboxylate | | |
| Propylene glycol | | 2.0% |
| Extract of cornflower | | 0.1% |
| Preserving agents | | 0.1% |
| PEG-60 hydrogenated castor oil | | 0.4% |
| Fragrance | | 0.1% |
| Water | qs | 100% |

1. Compounds of the 3-alkyl(4,5-diphenylimidazol-1-yl) family corresponding to formula (I) below: 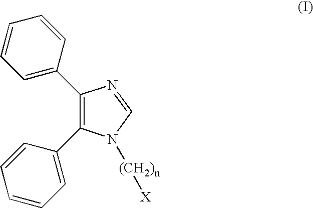
in which:
n is equal to 3, 4 or 5;
X represents:
(a) a radical —NR₁R₂ in which R₁ and R₂, which may be identical or different:
represent a saturated or unsaturated, linear or branched C₁-C₈ alkyl radical optionally substituted with at least one aryl radical, which is itself optionally substituted, and/or an aralkyl radical, which is itself optionally substituted, and/or a hydroxyl radical (—OH) and/or a radical —OR₄ and/or a radical —SR₄ and/or a radical —NR₄R₅, for which R₄ and R₅ represent a linear or branched C₁-C₄ alkyl radical,
form with the nitrogen atom an optionally substituted phthalimide,
form with the nitrogen atom a 5- or 6-membered ring optionally comprising at least one other hetero atom chosen from oxygen and/or nitrogen and/or sulphur;
(b) a radical —SR₃ or a radical —SOR₃ or a radical —SO₂R₃ or a radical —COR₃ or a radical —COOR₃ in which R₃ represents:
a linear or branched C₁-C₈ alkyl radical optionally substituted with at least one aryl and/or aralkyl radical and/or a hydroxyl radical (—OH) and/or a radical —OR₄ and/or a radical —SR₄ and/or a radical —NR₄R₅, for which R₄ and R₅ represent a linear or branched C₁-C₄ alkyl radical,
an aryl radical or an aralkyl radical or a heterocycle, optionally substituted with at least one alkyl radical and/or a hydroxyl radical (—OH) and/or a radical —OR₄ and/or a radical —SR₄ and/or a radical —NR₄R₅, for which R₄ and R₅ represent a linear or branched C₁-C₄ alkyl radical.

2. Compounds of formula (I) as defined in claim 1, characterized in that n is equal to 3 or 4.

3. Compounds of formula (I) as defined in any one of the preceding claims, characterized in that X represents a radical —NR₁R₂ or a radical —SR₃ or a radical —COR₃ or a radical —COOR₃.

4. Compounds of formula (I) as defined in any one of the preceding claims, characterized in that X represents a radical —NR₁R₂ in which R₁ and R₂ form with the nitrogen atom a 5- or 6-membered ring optionally comprising at least one other hetero atom chosen from oxygen and/or nitrogen and/or sulphur.

5. Compounds of formula (I) as defined in claim 4, characterized in that R₁ and R₂ form with the nitrogen atom a 6-membered ring.

6. Compounds of formula (I) as defined in either of claims 4 and 5, characterized in that the other hetero atom is an oxygen atom.

7. Compounds of formula (I) as defined in any one of claims 4 to 6, characterized in that R₁ and R₂ form with the nitrogen atom a morpholine.

8. Compounds of formula (I) as defined in any one of claims 1 to 4, characterized in that X represents a radical —NR₁R₂ in which R₁ and R₂ form with the nitrogen atom an optionally substituted phthalimide.

9. Compounds of formula (I) as defined in any one of claims 1 to 3, characterized in that x represents a radical —NR₁R₂ in which R₁ and R₂, which may be identical or different, represent a linear, saturated, unsubstituted C₁-C₄ alkyl radical.

10. Compounds of formula (I) as defined in claim 9, characterized in that the radicals R₁ and R₂ are identical.

11. Compounds of formula (I) as defined in. either of claims 9 and 10, characterized in that the radicals R₁ and R₂ are identical and represent a methyl radical or an ethyl radical.

12. Compounds of formula (I) as defined in any one of the preceding claims, chosen from:
3-phthalimidepropyl(4,5-diphenylimidazol)-1-yl,
4-phthalimidebutyl(4,5-diphenylimidazol)-1-yl,
5-phthalimidepentyl(4,5-diphenylimidazol)-1-yl,
4-[3-(4,5-diphenylimidazol-1-yl)propyl]morpholine,
5-[3.-(4,5-diphenylimidazol-1-yl)butyl]morpholine,
6-[3-(4,5-diphenylimidazol-1-yl)pentyl]morpholine,
4-[3-(4,5-diphenylimidazol-1-yl)propyl]thio-morpholine,
5-[3-(4,5-diphenylimidazol-1-yl)butyl]thio-morpholine,
6-[3-(4,5-diphenylimidazol-1-yl)pentyl]thio-morpholine,
4-[3-(4,5-diphenylimidazol-1-yl)propyl]piperidine,
5-[3-(4,5-diphenylimidazol-1-yl)butyl]piperidine,
6-[3-(4,5-diphenylimidazol-1-yl)pentyl]piperidine,
4-[3-(4,5-diphenylimidazol-1-yl)propyl]pyrrolidine,
5-[3-(4,5-diphenylimidazol-1-yl)butyl]pyrrolidine,
6-[3-(4,5-diphenylimidazol-1-yl)pentyl]pyrrolidine,
N,N′-diethyl-3-(4,5-diphenylimidazol-1-yl)-propylamine,
N-ethyl-N′-methyl-3-(4,5-diphenylimidazol-1-yl)-propylamine,
N,N′-dimethyl-4-(4,5-diphenylimidazol-1-yl)-butylamine,
N,N′-diethyl-4-(4,5-diphenylimidazol-1-yl)-butylamine,
N-ethyl-N′-methyl-4-(4,5-diphenylimidazol-1-yl)-butylamine,
N,N′-dimethyl-5-(4,5-diphenylimidazol-1-yl)-pentylamine,
N,N′-diethyl-5-(4,5-diphenylimidazol-1-yl)-pentylamine,
N-ethyl-N′-methyl-5-(4,5-diphenylimidazol-1-yl)-pentylamine,
N-methyl-N′-phenyl-3-(4,5-diphenylimidazol-1-yl)-propylamine,
N-ethyl-N′-phenyl-3-(4,5-diphenylimidazol-1-yl)-propylamine,
N-benzyl-N′-methyl-3-(4,5-diphenylimidazol-1-yl)-propylamine,
N-benzyl-N′-ethyl-3-(4,5-diphenylimidazol-1-yl)-propylamine,
N-methyl-N′-phenyl-4-(4,5-diphenylimidazol-1-yl)-butylamine,
N-ethyl-N′-phenyl-4-(4,5-diphenylimidazol-1-yl)-butyl amine,
N-benzyl-N′-methyl-4- (4,5-diphenylimidazol-1-yl)-butylamine,
N-benzyl-N′-ethyl-4-(4,5-diphenylimidazol-1-yl)-butylamine,
N-methyl-N′-phenyl-5-(4,5-diphenylimidazol-1-yl)-pentylamine,
N-ethyl-N′-phenyl-5-(4,5-diphenylimidazol-1-yl)-pentylamine,
N-benzyl-N′-methyl-5-(4,5-diphenylimidazol-1-yl)-pentylamine,
N-benzyl-N′-ethyl-5-(4,5-diphenylimidazol-1-yl)-pentylamine,
methyl (4,5-diphenylimidazol-1-yl)propyl-3-carboxylate,
methyl (4,5-diphenylimidazol-1-yl)butyl-4-carboxylate,
methyl (4,5-diphenylimidazol-1-yl)pentyl-5-carboxylate,
ethyl (4,5-diphenylimidazol-1-yl)propyl-3-carboxylate,
ethyl (4,5-diphenylimidazol-1-yl)butyl-4-carboxylate,
ethyl (4,5-diphenylimidazol-1-yl)pentyl-5-carboxylate,
3-thiomethylpropyl(4,5-diphenylimidazol)-1-yl,
4-thiomethylbutyl(4,5-diphenylimidazol)-1-yl,
5-thiomethylpentyl(4,5-diphenylimidazol)-1-yl,
3-thioethylpropyl(4,5-diphenylimidazol)-1-yl,
4-thioethylbutyl(4,5-diphenylimidazol)-1-yl,
5-thioethylpentyl(4,5-diphenylimidazol)-1-yl,
3-thiophenylpropyl(4,5-diphenylimidazol)-1-yl,
4-thiophenylbutyl(4,5-diphenylimidazol)-1-yl,
5-thiophenylpentyl(4,5-diphenylimidazol)-1-yl,
3-thiobenzylpropyl(4,5-diphenylimidazol)-1-yl,
4-thiobenzylbutyl(4,5-diphenylimidazol)-1-yl,
5-thiobenzylpentyl(4,5-diphenylimidazol)-1-yl,
3-methyloxopropyl(4,5-diphenylimidazol)-1-yl,
4-methyloxobutyl(4,5-diphenylimidazol)-1-yl,
5-methyloxopentyl(4,5-diphenylimidazol)-1-yl,
3-ethyloxopropyl(4,5-diphenylimidazol)-1-yl,
4-ethyloxobutyl(4,5-diphenylimidazol)-1-yl,
5-ethyloxopentyl(4,5-diphenylimidazol)-1-yl,
3-phenyloxopropyl(4,5-diphenylimidazol)-1-yl,
4-phenyloxobutyl(4,5-diphenylimidazol)-1-yl,
5-phenyloxopentyl(4,5-diphenylimidazol)-1-yl,
3-benzyloxopropyl(4,5-diphenylimidazol)-1-yl,
4-benzyloxobutyl(4,5-diphenylimidazol)-1-yl,
5-benzyloxopentyl(4,5-diphenylimidazol)-1-yl,
3-methylsulphonepropyl(4,5-diphenylimidazol)-1-yl,
4-methylsulphonebutyl(4,5-diphenylimidazol)-1-yl,
5-methylsulphonepentyl(4,5-diphenylimidazol)-1-yl,
3-ethylsulphonepropyl(4,5-diphenylimidazol)-1-yl,
4-ethylsulphonebutyl(4,5-diphenylimidazol)-1-yl,
5-ethylsulphonepentyl(4,5-diphenylimidazol)-1-yl,
3-phenylsulphonepropyl(4,5-diphenylimidazol)-1-yl,
4-phenylsulphonebutyl(4,5-diphenylimidazol)-1-yl,
5-phenylsulphonepentyl(4,5-diphenylimidazol)-1-yl,
3-benzylsulphonepropyl(4,5-diphenylimidazol)-1-yl,
4-benzylsulphonebutyl(4,5-diphenylimidazol)-1-yl,
5-benzylsulphonepentyl(4,5-diphenylimidazol)-1-yl,
3-methylsulphoxidepropyl(4,5-diphenylimidazol)-1-yl,
4-methylsulphoxidebutyl(4,5-diphenylimidazol)-1-yl,
5-methylsulphoxidepentyl(4,5-diphenylimidazol)-1-yl,
3-ethylsulphoxidepropyl(4,5-diphenylimidazol)-1-yl,
4-ethylsulphoxidebutyl(4,5-diphenylimidazol)-1-yl,
5-ethylsulphoxidepentyl(4,5-diphenylimidazol)-1-yl,
3-phenylsulphoxidepropyl(4,5-diphenylimidazol)-1-yl,
4-phenylsulphoxidebutyl(4,5-diphenylimidazol)-1-yl,
5-phenylsulphoxidepentyl(4,5-diphenylimidazol)-1-yl,
3-benzylsulphoxidepropyl(4,5-diphenylimidazol)-1-yl,
4-benzylsulphoxidebutyl(4,5-diphenylimidazol)-1-yl,
5-benzylsulphoxidepentyl(4,5-diphenylimidazol)-1-yl.

13. Compounds of formula (I) as defined in any one of the preceding claims, chosen from:
4-[3-(4,5-diphenylimidazol-1-yl)propyl]morpholine,
4-[3-(4,5-diphenylimidazol-1-yl)propyl]thio-morpholine,
4-[3-(4,5-diphenylimidazol-1-yl)propyl]piperidine,
methyl (4,5-diphenylimidazol-1-yl)propyl-3-carboxylate,
methyl (4,5-diphenylimidazol-1-yl)butyl-4-carboxylate,
3-phthalimidepropyl(4,5-diphenylimidazol)-1-yl,
4-phthalimidebutyl(4,5-diphenylimidazol)-1-yl.

14. Compounds of formula (I) as defined in any one of the preceding claims, chosen from:
4-[3-(4,5-diphenylimidazol-1-yl)propyl]morpholine,
4-[3-(4,5-diphenylimidazol-1-yl)propyl]piperidine.

15. Process for preparing the compounds of formula (I) as defined in any one of claims 1 to 14, characterized in that it consists essentially of the following steps:
a) dissolving 4,5-diphenylimidazole in an aprotic organic solvent preferably chosen from acetonitrile, acetone, tetrahydrofuran and dimethylformamide;
b) adding an organic or mineral base in an amount of between 1 and 10 equivalents;
c) adding an alkyl halide in an amount of between 1 and 5 equivalents;
d) heating to a temperature of between 60° C. and 85° C. for a period of between 6 hours and 48 hours;
e) evaporating the reaction medium to dryness under vacuum after cooling to room temperature;
f) dissolving the residue in water;
g) extracting the aqueous solution with an organic solvent chosen from ethyl acetate, dichloromethane and diethyl ether;
h) drying and evaporating the organic phase to dryness;
i) optionally purifying the residue.

16. Preparation process according to claim 15, characterized in that in step a), the aprotic organic solvent is acetonitrile.

17. Preparation process according to claim 15, characterized in that in step b), the base is a mineral base.

18. Preparation process according to either of claims 15 and 17, characterized in that in step b), the mineral base is chosen from potassium carbonate, calcium carbonate and sodium carbonate.

19. Preparation process according to any one of claims 15, 17 and 18, characterized in that in step b), the base is a mineral base and is potassium carbonate.

20. Preparation process according to any one of claims 15, 17 and 18, characterized in that in step b), between 1 and 3 equivalents and advantageously 2 equivalents of base are added.

21. Preparation process according to any one of claims 15 to 20, characterized in that in step c), 2 equivalents of alkyl halide are added.

22. Preparation process according to any one of claims 15 to 21, characterized in that in step d), the mixture is heated to the reflux point of the solvent.

23. Preparation process according to any one of claims 15 to 22, characterized in that in step d), the mixture is heated for a period of between 12 hours and 30 hours.

24. Preparation process according to any one of claims 15 to 23, characterized in that in step d), the mixture is heated for 24 hours.

25. Preparation process according to any one of claims 15 to 22, characterized in that in step g), the organic solvent is ethyl acetate.

26. Process for preparing the compounds of formula (I) as defined in any one of claims 1 to 14, characterized in that it consists essentially of the following steps:
a) dissolving 4,5-diphenylimidazole in a dipolar aprotic solvent chosen from dimethylformamide (DMF), dimethyl sulphoxide (DMSO) and dimethylacetamide (DMAc);
b) adding under an inert atmosphere an organic or mineral base in an amount of between 0.9 and 1.5 equivalents;
c) stirring the reaction medium at a temperature of between 25° C. and 100° C. for a period of between 30 minutes and 10 hours;
d) adding an alkyl halide in an amount of between 1 and 3 equivalents;
e) adding potassium iodide or sodium iodide in an amount of between 1 and 3 equivalents;
f) heating at a temperature of between 25° C. and 100° C. for a period of between 5 hours and 24 hours;
g) taking up the reaction medium in an organic solvent chosen from dichloromethane and chloroform;
h) washing the organic phase with water and bicarbonate, drying it and then evaporating it to dryness;
k) optionally purifying the residue.

27. Preparation process according to claim 26, characterized in that in step a), the aprotic organic solvent is DMF.

28. Preparation process according to either of claims 26 and 27, characterized in that in step b), the base is a mineral base.

29. Preparation process according to claim 28, characterized in that in step b), the mineral base is chosen from sodium hydride (NaH) and butyllithium (BuLi).

30. Preparation process according to either of claims 28 and 29, characterized in that in step b), the base is a mineral base and is sodium hydride.

31. Preparation process according to any one of claims 26 to 30, characterized in that in step b), between 1.0 and 1.1 equivalents and advantageously 1.0 equivalent of base is added.

32. Preparation process according to any one of claims 26 to 31, characterized in that in step c), the temperature is 50° C., for a period of between 1 hour and 2 hours, preferably for 1 hour.

33. Preparation process according to any one of claims 26 to 31, characterized in that in step d), between 1 and 2 equivalents of alkyl halide, advantageously 1.2 equivalents, are added.

34. Preparation process according to any one of claims 26 to 33, characterized in that in step e), between 1 and 2 equivalents of potassium iodide or sodium iodide, advantageously 1.2 equivalents, are added.

35. Preparation process according to any one of claims 26 to 34, characterized in that in step f), the mixture is heated at a temperature of 50° C., for a period of between 10 hours and 20 hours, preferably 15 hours.

36. Preparation process according to any one of claims 26 to 35, characterized in that in step g), the organic solvent is dichloromethane.

37. Composition comprising at least one compound of formula (I) as defined according to any one of claims 1 to 14.

38. Composition according to claim 37, characterized in that it is intended for cosmetic or pharmaceutical use.

39. Cosmetic composition according to either of claims 37 and 38, characterized in that it comprises at least one compound of formula (I) in an amount representing from 0.001% to 20% of the total weight of the composition and preferably in an amount representing from 0.01% to 5% of the total weight of the composition.

40. Composition according to any one of claims 37 to 39, characterized in that it also comprises at least one agent chosen from antibacterial agents, antiparasitic agents, antifungal agents, antiviral agents, antiinflammatory agents, antipruriginous agents, anaesthetics, keratolytic agents, free-radical scavengers, antiseborrhoeic agents, antidandruff agents, antiacne agents and/or agents for reducing cutaneous differentiation and/or proliferation and/or pigmentation, agents for improving the activity on regrowth of the hair and/or on stopping hair loss, and extracts of plant and/or bacterial origin.

41. Composition according to the preceding claim, characterized in that the antiinflammatory agents are chosen from steroidal or non-steroidal antiinflammatory agents.

42. Cosmetic or pharmaceutical composition, characterized in that it comprises, in a cosmetically or pharmaceutically acceptable medium, at least one compound of the 3-alkyl(4,5-diphenylimidazol-1-yl) family corresponding to formula (I) as defined in any one of claims 1 to 14 and at least one product with an irritant side effect.

43. Composition according to claim 42, characterized in that the product with an irritant side effect is chosen from ionic or nonionic surfactants, preserving agents, organic solvents or active agents, for instance α-hydroxy acids, β-hydroxy acids, α-keto acids, β-keto acids, retinoids, anthralins, anthranoids, peroxides, minoxidil, lithium salts, antimetabolites, vitamin D and its derivatives, hair dyes or hair colorants, fragrancing alcoholic solutions, antiperspirants, hair-removing active agents, permanent-waving active agents and depigmenting active agents.

44. Use, in a physiologically acceptable medium, in or for the preparation of a calmative composition, of at least one compound chosen from 4,5-diphenyl-1H-imidazole-1-propanamine, 4,5-diphenyl-1H-imidazole-1-butanamine and a compound of the 3-alkyl(4,5-diphenylimidazol-1-yl) family corresponding to formula (I) as defined in any one of claims 1 to 14.

45. Use according to claim 44, characterized in that the compound or the composition are intended for soothing skin disturbances chosen from sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes, particularly stinging, tingling, itching or pruritis, inflammation, discomfort and/or tautness.

46. Use, in a physiologically acceptable medium, in a cosmetic composition or for the preparation of a pharmaceutical composition, of at least one compound as defined in claim 44, the compound or the composition being intended to reduce and/or stabilize natural hair loss in man, advantageously androgenetic alopecia.

47. Cosmetic process for treating the skin and/or the scalp and/or mucous membranes, which is intended to soothe at least one of the skin disturbances chosen from sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes, characterized in that it consists in applying to the skin and/or the scalp and/or mucous membranes a cosmetic composition comprising at least one compound as defined in claim 44, leaving it in contact with the skin and/or mucous membranes and/or the scalp, and optionally rinsing it off.

48. Cosmetic treatment process according to claim 47, characterized in that it is intended for sensitive and/or irritable and/or reactive and/or intolerant skin and/or scalp and/or mucous membranes.

49. Cosmetic treatment process according to either of claims 47 and 48, characterized in that it is intended for the cosmetic treatment of natural hair loss in man.
